(19) United States
(12) Patent Application Publication
(10) Pub. No.: US 2019/0046356 A1
(43) Pub. Date: Feb. 14, 2019

(54) Methods Materials Assemblies Apparatuses and Implants for Surgical Reduction of Intraocular Pressure to Suprachoidal Space Ab Externo and Subconjunctival Space

(71) Applicant: Daniel Laroche, New York, NY (US)

(72) Inventor: Daniel Laroche, New York, NY (US)

(21) Appl. No.: 16/058,110

(22) Filed: Aug. 8, 2018

Related U.S. Application Data
(60) Provisional application No. 62/542,349, filed on Aug. 8, 2017.
(60) Provisional application No. 62/715,849, filed on Aug. 8, 2018.

(51) Int. Cl. A61F 9/007; A61F 9/00; A61F 2/14; A61M 27/00

(52) U.S. Cl. CPC A61F 9/00781; A61F 9/0017; A61K 9/0051; A61M 27/002; A61F 2/15

(57) ABSTRACT

Disclosed is an interocular fluid shunting assembly including a contact or interface plate and one or more fluid drainage tubes. According to embodiments there may be provided two tubes draining fluid to different locations. According to embodiments, a biodegradable polymer may be used to hinder fluid flow through at least one tube at initial insertion.

### RELATED APPLICATIONS

The present United States Utility Patent Application claims priority from Provisional Patent Applications Ser. No. 62/542,349, filed on the 8^{th} of Aug. 2017 and entitled: “Methods Materials Assemblies Apparatuses and Implants for Reducing Interocular Pressure”, and Ser. No. 62/715,849, filed on 8^{th} of Aug. 2018 and entitled “Methods Materials Assemblies Apparatuses and Implants For Surgical Reducing Intraocular Pressure to Suprachoidal Space Ab Externo and Subconjunctival Space”. All of the subject matter disclosed in and with the '349 and '849 provisional applications is hereby incorporated by reference in its entirety, including the complete disclosure of all the references cited therein.

### FIELD OF THE INVENTION

The present invention generally relates to the field of medical devices. More specifically, the present invention relates to methods, materials, assemblies, apparatuses and implants for reducing intraocular pressure, for example in persons or others subjects with glaucoma.

### BACKGROUND

Intraocular pressure in the eye is maintained by the formation and drainage of aqueous, a clear, colorless fluid that fills the anterior and posterior chambers of the eye. Aqueous normally flows from the anterior chamber of the eye out through an aqueous outflow channel at a rate of 2 to 5 microliters per minute. Glaucoma is a progressive disease of the eye characterized by a gradual increase of intraocular pressure. This increase in pressure is most commonly caused by stenosis or blockage of the aqueous outflow channel known as the trabecular meshwork, resulting in excessive buildup of aqueous fluid in the eyeball, Other causes include increase in venous pressure outside the eye which is reflected back through the aqueous drainage channels and increased production of aqueous. In a “normal” eye, intraocular pressure mean is about 15 mm mercury. In an eye with glaucoma, this pressure can range between the so-called normal pressures and pressures of up to as much as 50 mm mercury. This increase in intraocular pressure produces gradual and permanent loss of vision in the afflicted eye due to increased mechanical stress on the optic nerve.

Existing corrective methods for the treatment of glaucoma include drugs, surgery, and implants. Pharmacological treatment is prohibitively expensive to a large majority of glaucoma patients. In addition, many people afflicted with the disease live in remote or undeveloped remote areas where the drugs are not readily accessible. The drugs used in the treatment, in particular steroids, often have undesirable side effects and many of the long-term effects resulting from prolonged use are not yet known.

Surgical procedures have been developed in an effort to treat patients with glaucoma. An iridectomy, removal of a portion of the iris, is often used in angle-closure glaucoma wherein there is an occlusion of the trabecular meshwork by iris contact. Removal of a piece of the iris then gives the aqueous free passage from the posterior to the anterior chambers in the eye. A trabeculectomy, opening the inner wall of Schlemm's canal is often performed in cases of developmental or juvenile glaucoma so as to increase the outflow of the aqueous, thereby decreasing intraocular pressure. In adults, a trabeculectomy shunts fluid through a trap-door flap in the eye that performs a valve-like function for the first few weeks after surgery. While often successful, these surgical techniques possess inherent risks associated with invasive surgery on an already afflicted eye. Furthermore, the tissue of the eye can scar over this small area and the eye reverts to the pre-operative condition, thereby necessitating the need for further treatment.

Ocular implants are often used in long-term glaucoma treatment. One early implant was invented by Dr. Anthony Molteno and is described in the paper entitled “Use of Molteno Implants to Treat Secondary Glaucoma” by A. C. B. Molteno and published by Grune & Stratton, Ltd, 1986, pp 211-238, which is hereby incorporated by reference in its entirety. The implant was a small circular plate with a rigid trans limbal drainage tube attached thereto. The plate was 8.5 mm in diameter and formed a surface area of 48 mm². This early Molteno implant was sutured to the sclera in the anterior segment of the eye at the limbus and the drainage tube was inserted into the anterior chamber of the eye. Once implanted, the body forms scar tissue around this plate. Increased pressure causes the tissues above the plate to lift and form a bleb into which aqueous flows from the anterior chamber via the drainage tube. Many problems occurred with the early Molteno device. The bleb that formed around the plate, even though it was small in surface area on the sclera, formed a very tall bleb which resulted in Dellen formation (sterile corneal ulcers). The implant had to be removed in another surgery to cure the ulcers. Further, as indicated at page 220 of Dr. Molteno's paper, his early device did not reduce the intraocular pressure enough to treat the glaucoma without the use of additional medications. Dr. Molteno redesigned his implant for insertion into the posterior segment of the eye to avoid the problems with his early anterior device.

The redesigned Molteno implant is disclosed in U.S. Pat. No. 4,457,757 entitled “Device for Draining Aqueous Humor,” which is hereby incorporated by reference in its entirety. This implant is commercially available as the Molteno™ Seton implant and also referred to as the long tube Molteno implant. As indicated at page 221 of Dr. Molteno's paper, the long tube Molteno implant has been used exclusively by Dr. Molteno since 1973. The implant comprises a flexible drainage tube connected to one or more rigid plate reservoirs. The plates are shaped to conform to the curvature of the eye. The long tube Molteno implant is disadvantageous as the plates are formed of a rigid plastic which makes insertion beneath the eye tissue difficult and time-consuming. The plates are 13 mm in diameter and therefore the bleb formation area is at least 134 mm2. The reservoir plate is placed under Tenon's capsule in the posterior segment of the eye and sutured to the sclera. The drainage tube is implanted into the anterior chamber through a scleral flap. A second plate can be passed under or over the superior rectus muscle also in the posterior segment of the eye and sutured to the sclera. In the redesign of the Molteno implant, Dr. Molteno not only moved the bleb forming part, or plate, of the implant back to the posterior segment of the eye, he also increased the bleb formation area from at least 48 to at least 134 mm2, because it was believed that there was not enough room to form a large bleb in the anterior segment of the eye.

U.S. Pat. No. 4,750,901 issued to Molteno, which is hereby incorporated herein by reference, discloses another glaucoma implant with an elevated peripheral ridge, a subsidiary elevated ridge on the upper surface of the implant and a drainage tube which leads from the upper surface of the plate to the anterior chamber of the eye. This device is also implanted in the posterior segment of the eye under Tenon's tissue, i.e., Tenon's capsule. This Molteno patent discloses that the tube enters the peripheral ridge to a position above the upper surface of the plate and the subsidiary ridge is located around the entrance of the tube. The subsidiary ridge is forced against Tenon's capsule, to create an initial bleb cavity much smaller in area than the total bleb cavity, but both cavities are formed in the posterior segment of the eye. This Molteno patent discloses that the addition of the subsidiary ridge to the upper surface of the plate around the exit of the tube has the effect of providing a pressure sensitive one-way valve effect. The Molteno patent also discloses that, once the eye recovers from the operation, the increased production of aqueous fluid by the eye raises the pressure in the eye and also within the small bleb cavity causing the overlying Tenon's capsule to be lifted slightly, thereby allowing fluid to flow into the entire bleb cavity. In practice however, the Molteno device fails to provide an effective sealing surface with Tenon's capsule and the desired one-way valve effect does not occur.

UK Patent Application 2,160,778 entitled “Aqueous humor drainage device” discloses a similar type of implant device comprising a drainage tube and a drainage body. The tube is fixed to and opens directly onto a surface of the body. The device is sutured to the sclera of the eye in the posterior segment of the eye and the tube positioned within the anterior chamber to provide outflow for the aqueous contained therein.

U.S. Pat. No. 4,729,761 discloses a Glaucoma implant with a plate, a separate fluid reservoir, a first tube between the plate and the fluid reservoir and a second tube between the fluid reservoir and the anterior chamber of the eye. The plate is attached to the sclera in the posterior segment of the eye, and a bleb forms around the plate. Despite the more anterior location of the fluid reservoir, the housing around which the bleb is formed is located in the posterior segment of the eye.

Another glaucoma implant device called the Optimed Glaucoma Implant made by Optimed, Inc. of Santa Barbara, Calif. This implant comprises a box valve connected to a drainage tube which extends into the anterior chamber of the eye. The box valve has a dimension of approximately 3 mm×2 mm×2 mm and has a maximum top surface area of 18 mm2. The box has a small extension with holes therein to form suture locations to attach the box to the eye. The box contains approximately 180-200 microtubules which are attached to one end of the drainage tube. The microtubules act like a valve to limit the flow of aqueous humor from the anterior chamber. The drainage tube is implanted into the anterior chamber of the eye. The box and housing is sutured to the sclera in the anterior segment close to the limbus. The housing does not constitute a plate or a drainage surface.

Another glaucoma implant is the Ahmed Valve patent number U.S. Pat. No. 5,071,408 A. This is a medical device composed of a pair of plates holding in tension a membrane folded over to form a chamber with an elongated, slit-like opening adjoining edges. The membrane is designed to open at a pressure of 9 mmHg. However shortly after placement an ocular hypertensive phase often occurs leading to higher pressures and the need for additional medications or surgery.

Another glaucoma implant is the Baerveldt implant, patent number U.S. Pat. No. 6,050,970 A. The implant comprises an elastomeric plate having first and second surfaces and a non-valved elastomeric drainage tube. The plate is positioned over a sclera of said eye beneath Tenon's capsule, such that a portion of the plate extends into the anterior segment of the eye. The first end of said elastomeric drainage tube is open to said second surface of said plate. The second end of said drainage tube is tunneled through the sclera and cornea and inserted into the anterior chamber of said eye. Fluid communication is provided between said anterior chamber and a scar tissue bleb which forms around said implant. Preferably, a portion of the scar tissue bleb extends into the anterior segment of the eye and a portion of the scar tissue bleb extends into the posterior segment of the eye. The tube is larger and must be tied off. There is no immediate lowering of intraocular pressure. Hypotony is a major issue due to the larger ostium of the tube insertion into the anterior chamber. The plate is larger necessitating placement under the muscles increasing the likelihood of motility disorders.

A listing of additional publications relating the subject-matter of the present application are included in an Information Disclosure Statement (IDS) filed concurrently with the this application. All teachings of all of the above cited publications and those publications listed in the IDS filed herewith are hereby incorporated by reference in their entirety.

### SUMMARY OF INVENTION

The present invention may include methods, materials, assemblies, apparatuses and implants for reducing intraocular pressure within a subject's eye. According to embodiments of the present invention, there may be provided an implantable assembly suitable for implanting onto and within an eye to be treated. The implantable assembly according to embodiments of the present invention may include at least one variable flow rate fluid shunts or tubes to provide for drainage of aqueous humor from a respective eye. According to embodiments of the present invention, the implant may comprise an elastomeric plate having first and second surfaces and two non-valved elastomeric drainage tubes. The plate may be positioned over a sclera of an eye to be treated, for example above Tenon's capsule, such that a portion of the plate extends into the anterior segment of the eye. The first end of one of said elastomeric drainage tube may be open to said second surface of said plate. The second end of one said drainage tube may be tunneled through the sclera and cornea and inserted into the anterior chamber of said eye. Fluid communication may be provided between said anterior chamber and a tissue bleb which forms around said implant. Preferably, a portion of the tissue bleb extends into the anterior segment of the eye and a portion of the tissue bleb extends into the posterior segment of the eye. A second end of the said elastomeric drainage tube runs parallel to the first tube and inserted into the anterior chamber of the eye. The posterior end of the second tube runs posteriorly to drain aqueous to an inferior compartment. This will allow drainage to a more posterior tube compartment and to a choroidal window that is dissected beneath the aqueous drainage device, and to a posterior tube to drain posteriorly. Both elastomeric drainage tubes that are inserted into the anterior chamber dramatically after the biomechanical properties of the eye reducing scleral resistance and stress on the optic nerve. Drainage is directed to form an anterior bleb, suprachoroidal absorption, and a posterior bleb.

The present invention provides an implant for the treatment of glaucoma having a bleb formation device and a draining tube, wherein at least a portion of the bleb formation device can be implanted in the anterior segment of the eye, thereby causing a portion of the tissue bleb to be formed in the anterior segment of the eye. The remainder of the bleb is formed in the posterior segment of the eye. There is also an inferior compartment to drain aqueous to the suprachoroidal space via a scleral window created beneath the plate. The scleral window reduces biomechanical stress on the optic nerve and offers a unique additional outflow.

In one embodiment, the implant creates a temporary seal to restrict the flow of fluid from the anterior chamber of the eye and after a period of time provides flow between the larger surface around the implant and the anterior chamber of the eye. The implant comprises a single plate formed of a pliable, elastomeric material having two non-valved tube attached to and opening onto a surface of the plate. The plate is sutured to the scleral tissue in the anterior segment of the eye at the forward portion of the plate utilizing permanent sutures to keep the plate from migrating and impinging on the eye socket tissue or extruding from the eye tissue.

The attached plate is covered by the conjunctiva and forms a drainage bleb. The attached tubes are tunneled through the sclera and the cornea and positioned within the anterior chamber to provide a drain for aqueous fluid within the eye. Because of the pliable construction and smaller size, the device can be implanted with greater ease than previous implants. This may substantially shorten the time required to perform the surgical procedure and to implant such large surface area implants. The second tube allows for further drainage to lower intraocular pressure and drains to the posterior portion of the eye. One or both tubes can be tied off to delay fluid outflow depending on individual patient circumstances.

In one embodiment of the present invention, the method of treating glaucoma in an eye is performed utilizing an implant. The implant comprises a bleb formation device and an elastomeric drainage tube, wherein a first end of the elastomeric drainage tube is open to a surface of the bleb formation device. The bleb formation device is positioned in the eye. A drainage bleb forms around the bleb formation device, such that at least a portion of the bleb is formed in the anterior segment of the eye. The second end of the drainage tube is positioned within the anterior chamber of the eye thus providing fluid communication between the anterior chamber and the bleb. Preferably, the bleb formation device is positioned over the sclera and/or beneath Tenon's capsule of the eye.

According to additional embodiments, a second tube bypasses the bleb to allow additional drainage to the posterior aspect of the eye. The second tube may overcome any hypertensive phase in the post-operative period that can lead to failure. The second tube may also provide additional drainage.

In another embodiment of the method of treating glaucoma utilizing an implant, wherein the implant comprises a bleb formation device and an elastomeric drainage tube, the bleb formation device is positioned in the eye, such that at least a portion of the bleb formation device is anterior to the muscle insertions of the eye. The second end of the drainage tube is positioned within the anterior chamber of the eye, and fluid communication is provided between the anterior chamber and a bleb which forms around the implant. The bleb formation device may be positioned over the sclera and/or above Tenon's capsule of the eye. The bleb formation device may be an elastomeric plate, wherein the first end of the tube is open to a surface of the elastomeric plate. The bleb formation device may be sutured to the eye and to the sclera of the eye. In an embodiment, the bleb formation device is positioned such that between 5% and 10% of the bleb formation device is anterior to the muscle insertions of the eye. The bleb formation device may be positioned such that between 85% of the bleb formation device is in between the muscle insertions of the eye.

In a preferred embodiment, the implant for draining aqueous fluid from a first region of an eye to a second region of the eye which includes the sclera comprises an elastomeric plate and a drainage tube. Preferably, the first region of the eye may be an anterior chamber of the eye and the second region of the eye may be an outside region of the eye. The elastomeric plate has first and second surfaces to conform to the second region of the eye, the outer surface of the eye. The drainage tube may include a first end and a second end. The drainage tube may be attached to the plate such that the first end of the drainage tube opens onto the second surface of the elastomeric plate. The second end of the drainage tube may be in fluid communication with the first region of the eye. Preferably, the drainage tube is less than 10 mm in length. More preferably, the drainage tube is less than 8 mm in length.

According to embodiments, there may be provided an interocular fluid shunting assembly comprising an interface plate including one or more latching structures configured to facilitate securing of said plate to an outer surface of an eyeball. There may be one or more fluid tubes supported by the plate, wherein each of said one or more fluid tubes includes first and second ends, at least one of which tube ends may be adapted to penetrates an outer surface of an eyeball to a depth sufficient for contact with interocular fluid. There may be a fluid flow hindering element of biodegradable material functionally associated with at least one of the one or more fluid tubes and adapted to hinder interocular fluid flow through the functionally associated fluid tubes upon initial penetration of the tubes into the eyeball.

A surface of said plate intended for contact with an outer surface of an eyeball may be of a partial spherical shape. The surface may include latching structures configured to facilitate securing of said plate to the outer surface of an eyeball. The latching structures may include one or more structure selected from the group consisting: (a) one or more suturing holes, (b) toothlike protrusions; and (c) surface segments with textures configured to promote bleb formation.

One or more tubes of the assembly may pass through said plate. The flow hindering element functionally associated with at least one of the fluid tubes may be a biodegradable polymer clog located within an inner channel of the tube. The flow hindering element functionally associated with at least one of the fluid tubes may be a pinching element located outside the tube and physically pinches the tube. The pinching element is a biodegradable suture wrapped around the tube.

As assembly according to embodiments may include two fluid shunting tubes. One of the tubes may be associated with a fluid hindering element and the other may not. Each of the tubes may drain interocular fluid to either the same or to a different location.

According to embodiments, the two tubes may provide for subconjunctival and suprachoroidal flow. The tubes may provide for ab externo suprachoriodal outflow with one tube end accessing the aqueous and other tube end inserted to suprachoroidal space ab externo.

### BRIEF DESCRIPTION OF THE FIGURES

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a set of mechanical drawings of the constituent elements of an implant in accordance with embodiments of the present invention;

FIGS. 2A & 2B are illustrations of an implant according to embodiments of the present invention implanted onto and within an eye to be treated by the implant;

FIGS. 3A & 3B are prospective views of a shunt assembly in accordance with embodiments of the present invention; and

FIG. 3C is a prospective view of the shun assembly disassembled.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE FIGURES

In the following detailed description, numerous specific details are set forth in order to provide a thorough understanding of the invention. However, it will be understood by those skilled in the art that the present invention may be practiced without these specific details. In other instances, well-known methods, procedures, components and assemblies have not been described in detail so as not to obscure the present invention.

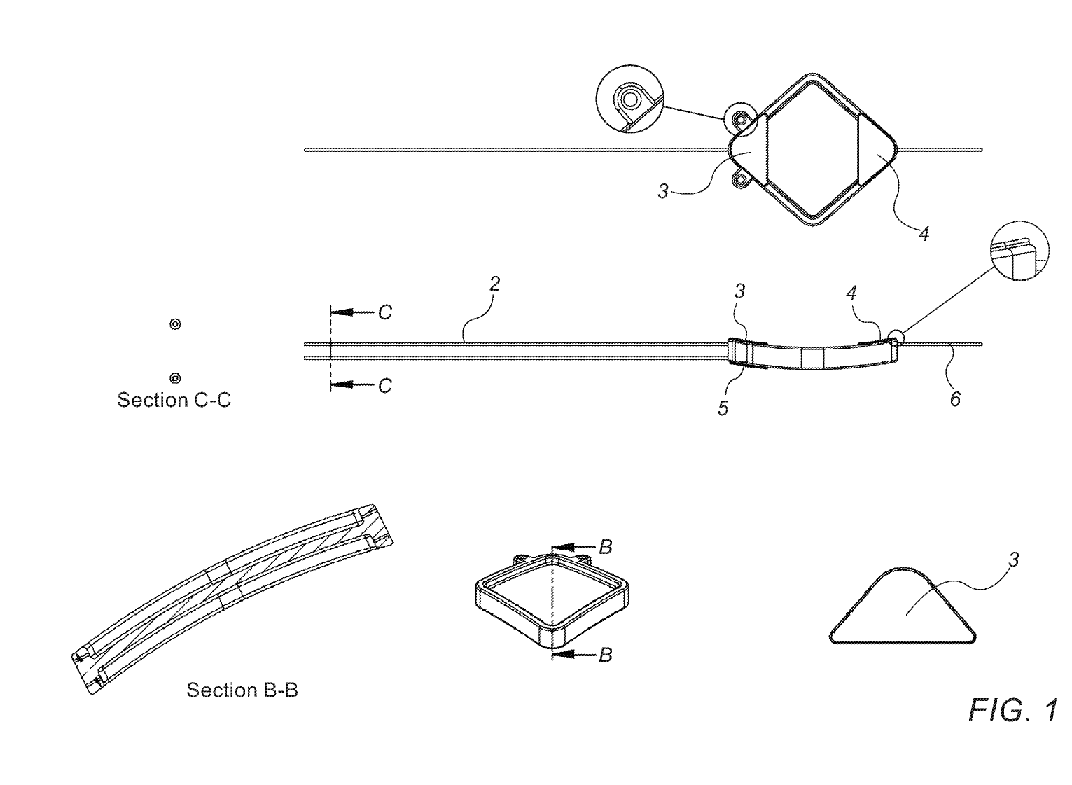

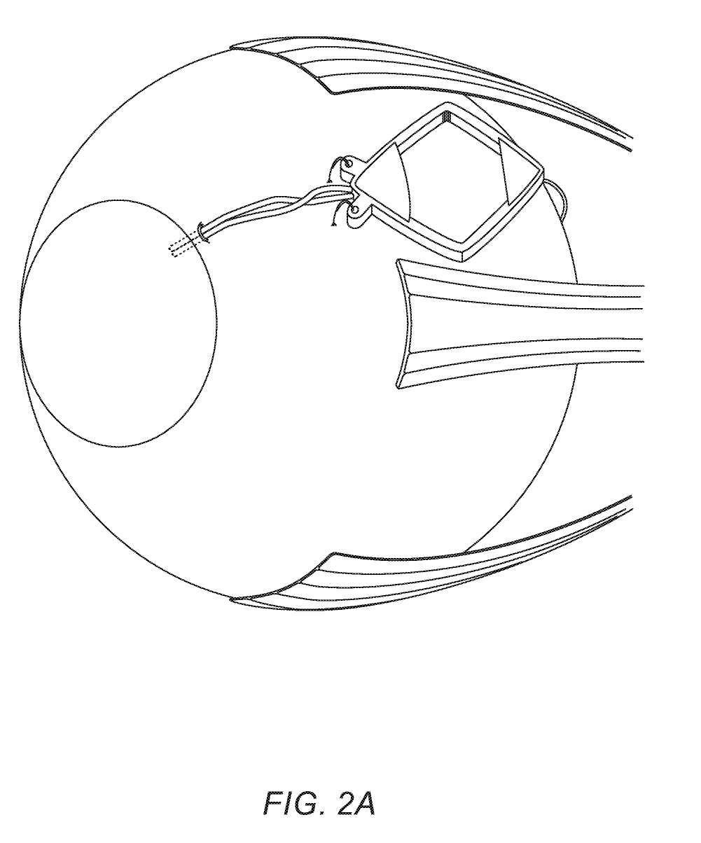

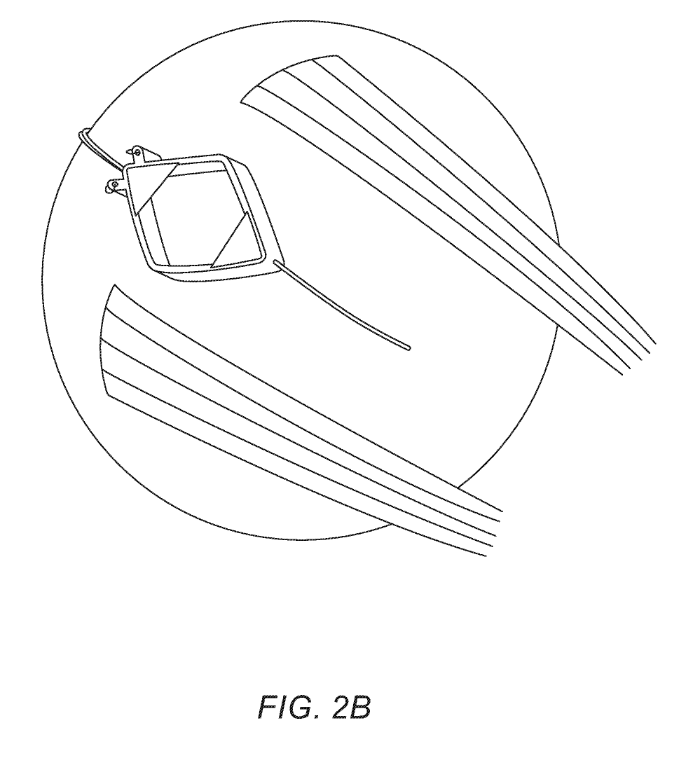

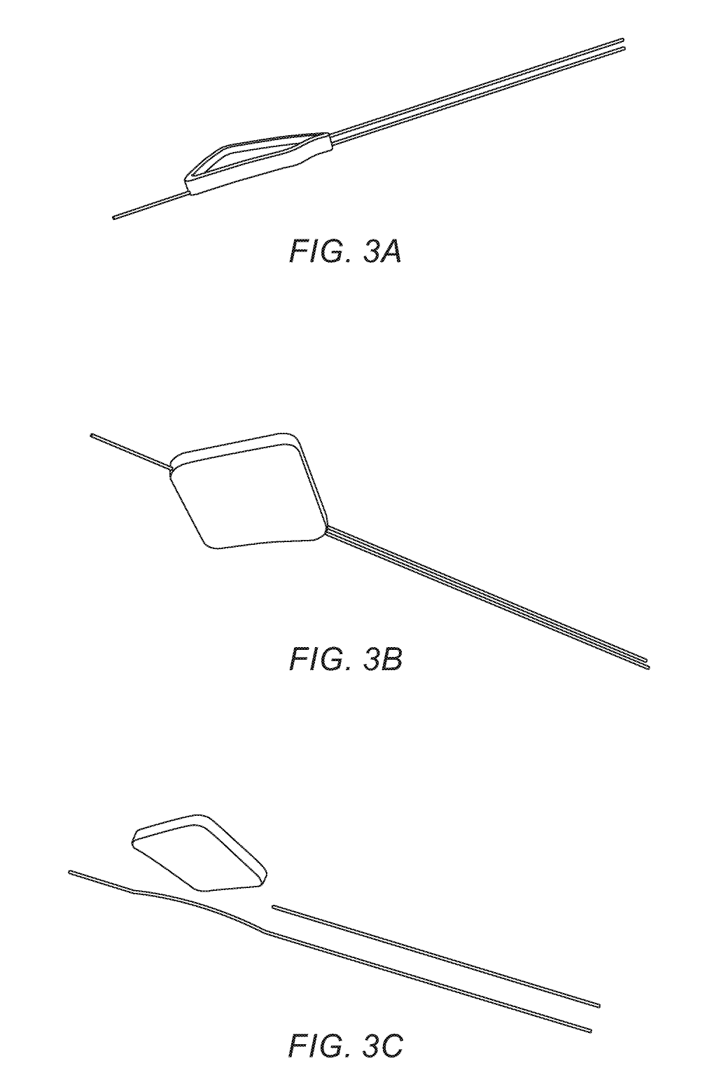

The following description is made with reference to FIG. 1, FIGS. 2A & 2B and FIGS. 3A through 3C. The relevant structure of the eye will be described briefly below to provide background for the anatomical terms incorporated herein, however, it should be realized that several anatomical details have been omitted for clarity of understanding. The tough outer membrane known as the sclera covers all of the eye except that portion covered by the cornea, the thin, transparent membrane which covers the iris and the pupil. The cornea merges into the sclera at a juncture referred to as the sulcus of the sclera or as the limbus. A portion of the sclera is covered by a thin tissue called the conjunctiva. The ciliary body begins at the limbus and extends along the interior of the sclera and becomes the choroid. The choroid is a brown vascular membrane which extends along the retina back toward the optic nerve. The suprachoroidal space also serves as an additional drainage site of aqueous. The eye includes six extraocular eye muscles which control the movement of the eye in the socket. The eye muscles include the rectus muscles which include lateral, medial, superior, oblique and inferior muscles. The muscle insertion is the point at which the rectus muscles attach to the globe of the eye. The anterior segment is the portion of the globe of the eye which is anterior to the muscle insertions. The remainder of the globe which is posterior to the muscle insertions is considered the posterior segment.

It is well-known that aqueous is produced by the ciliary body and reaches the anterior chamber formed between the iris and the cornea through the pupil. In a normal eye, the aqueous is removed through the trabecular meshwork. There the aqueous passes through Schlemm's canal and through veins which merge with blood-carrying veins and into venous circulation. Intraocular pressure is maintained in the eye by the intricate balance of secretion and absorption or outflow of the aqueous in the manner described above. Glaucoma results from excessive buildup of aqueous fluid in the anterior chamber which produces an increase in intraocular pressure.

Implants for treatment of glaucoma facilitate the outflow of the aqueous from the anterior chamber of the eye. The implant comprises a pliable plate, also referred to as a pliable seton in the ophthalmic field, having oppositely disposed first and second curved surfaces, connected to two drainage tubes which extends into a first region of the eye. The seton is implanted in a second region of the eye above or beneath a layer of Tenon's capsule and sutured to the sclera. More specifically, the implant is implanted in the posterior segment of the eye. The tubes will redirect aqueous to the subconjunctival space and posterior sclera. When a scleral window is dissected beneath the plate flow will also be directed to the suprachoriodal space facilitating additional outflow. The scleral window will also further reduce biomechanical stress to the optic nerve.

The surface area of the plate is preferably in the range of approximately 37 mm2 on each side and fits between the muscles.

The Plate is 4 mm×4 mm made of medical grade silicone elastomer. There is a partial cover on the superior and inferior apex. A superior and inferior tube are inserted at the apex. The superior tube connects to a superior chamber to transport aqueous to the subconjunctival space. The inferior tube connects to the inferior plate and transports to the inferior chamber that connects to both a scleral window that is dissected to the suprachoroidal space. Aqueous fluid can then drain to the suprachoroidal space and to a posterior tube connected at the back portion of the plate. The posterior tube drains aqueous to a posterior bleb that is formed separately from the subconjunctival bleb. The internal opening of each tube is 70 u allowing flow restriction to reduce the risk of hypotony. There is a PMMA plate at both the superior and inferior apex to prevent obstruction of the opening.

The implant can be inserted into the posterior segment of the eye using known ophthalmological surgical techniques. An initial incision is made in the conjunctiva and Tenon's capsule parallel to the limbus, the incision is stretched to enable the insertion of the implant. Just where the plate is to be inserted a 1.5 mm·1.5 mm scleral widow to the choroid can be dissected. The plate is inserted into the second region of the eye above the scleral window, beneath the muscles superotemporally. The plate can be sutured to the sclera. Preferably, nonabsorbable nylon sutures are used in the suture holes to secure the plate or 9-0 nylon or polypropylene sutures. The drainage tube is tunneled out through the sclera and the cornea beneath Tenon's capsule and in through an incision in the region of the limbus such that the second end of the tube extends into a first region, such as the anterior chamber, of the eye of a phatic eye. The second tube can be inserted in the same manner as the first. Both tubes can be inserted in the posterior chamber pseudophakic eyes. The exposed portion of the drainage tube is then covered with a partial thickness auto scleral graft adjacent to the tube. Or scleral reinforcing element. The drainage tube may be closed with a temporary suture(s) to prevent any drainage of aqueous prior to formation of the bleb tissue over the plate. Or only one of the tubes can be tied off. In actual practice, it has been found that initially after surgery aqueous fluid will weep through a space formed between the yet to be healed incision and the drainage tube. This weeping of the aqueous fluid through the incision relieves some of the fluid pressure until the bleb has formed and the temporary suture(s) is/are removed or absorbed by the body. In one embodiment, the temporary suture(s) can be s a dissolvable suture while suture is nonabsorbable. In an alternate, but not preferred, embodiment, the temporary sutures are removed during a secondary procedure, such as a surgical procedure or an ophthalmic laser procedure. Both procedures are known to those of skill in the art.

The formation of the bleb occurs in response to the introduction of the plate into the tissue of the second region of the eye. The bleb comprises a thin layer of connective tissue which encapsulates the plate, and substantially all of the surfaces of the plate contact the tissues in the second region of the eye, thus lifting the Tenon's capsule above the sclera as shown. Typically, bleb formation occurs in the range of 1 to 8 weeks postoperatively. A dissolving suture can be used to seal the drainage tube. After removal or dissolution of the suture(s) blocking the drainage tube the aqueous flow between the tube and bleb is advantageously a patent flow, allowing both flow from the anterior chamber to the bleb, to suprachoroidal window and the posterior segment. This ensures that retrograde non-valved flow from the bleb to the anterior chamber, occurring in response to pressure on the eye from the outside, for example, when the lid is forced closed or when the eyeball is pressed on with a finger, does not adversely or harmfully affect intraocular pressure within the eye. The fluid contained in the bleb seeps through the bleb into intercellular spaces within the eye and is then removed through surrounding capillaries or lymphatics.

Advantageously, the insertion of the implant, such that a portion of the plate extends into the anterior segment, provides for a simpler insertion procedure. It is easier to suture the plate to the sclera when the suture holes are located in the anterior segment of the eye. Finally, by having anterior and posterior drainage areas on the plate the surface area of the plate can be increased. Thus, a plate can be formed to cover the sclera in the anterior segment and posterior segment of the eye. By increasing the surface area of the plate, the surface area of the bleb that forms around the plate increases. It has been shown that by increasing the surface are of the bleb that covers the sclera, increased uveoscleral outflow and reducing biomechanical scleral stress, the intraocular pressure can be decreased more significantly. Thus, this implant with an increased surface area, greater opportunity for outflow will be more effective at treating the most severe cases of glaucoma without requiring additional medications.

The flexible elastomeric material used to form parts of the present invention, and the size and elliptical shape of the plate allows the implant to be inserted much more easily than previously realized with other glaucoma treatment implants. Further, the flexible material from which the plate 38 is formed is soft and pliable which results in much less trauma and irritation to the surrounding tissues and vasculature than experienced with a rigid plate device. Thus, the pliable plate 38 significantly decreases the surgical procedure length while also minimizing tissue and vasculature damage which can occur in the insertion process.

In the preferred embodiment of the implant the plate has a profile shape that is generally spherical and conforms to the contour of the eye. Preferably, the plate is shaped like the profile of an elongated soybean. The square shape allows for symmetrical drainage of flow within the muscle space to reduce diplopia and bleb formation over the muscles and direct the flow more posteriorly.

The drainage tube has an inner diameter of 70 um and an outer diameter of 150 um. The drainage tube of the preferred embodiment 25 mm for both anterior tubes that can be cut depending on the size of the eye and 5 mm posterior.

Preferably, the temporary sutures are dissolvable sutures. The sutures can be absorbed at any time postoperatively from 1 day up to 8 weeks. Preferably, the sutures are dissolved in 1 to 6 weeks postoperatively. In the preferred embodiment, the dissolving sutures are 7-O or 8-O Vicryl. after the bleb forms, the temporary sutures are absorbed by the body, the fluid pressure in the cupped cavity drains to the suprachoroidal window, subconjunctival space and posterior portion of the eye.

As indicated above, the temporary sutures of the preferred embodiment are dissolvable sutures. In an alternate, but not preferred embodiment, the temporary sutures are removed during a secondary procedure, such as a surgical procedure or a laser procedure, after scar tissue formation. In the preferred embodiment, the temporary sutures are selected such that the sutures dissolve after the formation of the scar tissue bleb. The sutures can be absorbed at any time postoperatively from 1 day up to 8 weeks. Preferably, the sutures are dissolved in 1 to 6 weeks postoperatively. In the preferred embodiment, the dissolving sutures are 8-O Vicryl. Aqueous fluid moves posteriorly to a superior bleb, inferior container that connects to a scleral window to provide suprachoridal drainage, and the also to a posterior tube to form a more posterior bleb. Increasing the avenues for fluid drainage will reduce IOP to lower targets of 10-12 mmHG to prevent blindness from glaucoma and also allow improvement of vision from damaged retinal ganglion cells that are not dead yet. This implant with an increased surface area will be more effective at treating the most severe cases of glaucoma without requiring additional medications.

According to embodiments, there may be provided an interocular fluid shunting assembly comprising an interface plate including one or more latching structures configured to facilitate securing of said plate to an outer surface of an eyeball. There may be one or more fluid tubes supported by the plate, wherein each of said one or more fluid tubes includes first and second ends, at least one of which tube ends may be adapted to penetrates an outer surface of an eyeball to a depth sufficient for contact with interocular fluid. There may be a fluid flow hindering element of biodegradable material functionally associated with at least one of the one or more fluid tubes and adapted to hinder interocular fluid flow through the functionally associated fluid tubes upon initial penetration of the tubes into the eyeball.

A surface of said plate intended for contact with an outer surface of an eyeball may be of a partial spherical shape. The surface may include latching structures configured to facilitate securing of said plate to the outer surface of an eyeball. The latching structures may include one or more structure selected from the group consisting: (a) one or more suturing holes, (b) toothlike protrusions; and (c) surface segments with textures configured to promote bleb formation.

One or more tubes of the assembly may pass through said plate. The flow hindering element functionally associated with at least one of the fluid tubes may be a biodegradable polymer clog located within an inner channel of the tube. The flow hindering element functionally associated with at least one of the fluid tubes may be a pinching element located outside the tube and physically pinches the tube. The pinching element is a biodegradable suture wrapped around the tube.

As assembly according to embodiments may include two fluid shunting tubes. One of the tubes may be associated with a fluid hindering element and the other may not. Each of the tubes may drain interocular fluid to either the same or to a different location.

According to embodiments, the two tubes may provide for subconjunctival and suprachoroidal flow. The tubes may provide for ab externo suprachoriodal outflow with one tube end accessing the aqueous and other tube end inserted to suprachoroidal space ab externo.

Although the invention has been described with reference to specific embodiments, the description is intended to be illustrative of the invention and is not intended to be limiting. Various modifications and applications may occur to those skilled in the art without departing from the true spirit and scope of the invention as defined in the appended claims.

Functions, operations, components and/or features described herein with reference to one or more embodiments, may be combined or otherwise utilized with one or more other functions, operations, components and/or features described herein with reference to one or more other embodiments, or vice versa. While certain features of the invention have been illustrated and described herein, many modifications, substitutions, changes, and equivalents will now occur to those skilled in the art. It is, therefore, to be understood that the appended claims are intended to cover all such modifications and changes as fall within the true spirit of the invention.

1. An interocular fluid shunting assembly comprising:
an interface plate including one or more latching structures configured to facilitate securing of said plate to an outer surface of an eyeball;
one or more fluid tubes supported by said plate, wherein each of said one or more fluid tubes includes first and second ends, at least one of which tube ends is adapted to penetrates an outer surface of an eyeball to a depth sufficient for contact with interocular fluid;
a flow hindering element of biodegradable material functionally associated with at least one of the one or more fluid tubes and adapted to hinder interocular fluid flow through the functionally associated fluid tubes upon initial penetration of the tubes into the eyeball.

2. The interocular fluid shunting assembly of claim 1, wherein a surface of said plate intended for contact with an outer surface of an eyeball is of a partial spherical shape.

3. The interocular fluid shunting assembly of claim 2, wherein said latching structures configured to facilitate securing of said plate to an outer surface of an eyeball include one or more structure selected from the group consisting: (a) one or more suturing holes, (b) toothlike protrusions; and (c) surface segments with textures configured to promote bleb formation.

4. The interocular fluid shunting assembly of claim 1, wherein said one or more tubes pass through said plate.

5. The interocular fluid shunting assembly of claim 1, wherein the flow hindering element functionally associated with at least one of the fluid tubes is a biodegradable polymer clog located within an inner channel of the tube.

6. The interocular fluid shunting assembly of claim 1, wherein the flow hindering element functionally associated with at least one of the fluid tubes is a pinching element located outside the tube and physically pinches the tube.

7. The interocular fluid shunting assembly of claim 6, wherein the pinching element is a biodegradable suture wrapped around the tube.

8. The interocular fluid shunting assembly of claim 1, including two fluid shunting tubes.

9. The interocular fluid shunting assembly of claim 8, wherein one of the tubes is associated with a fluid hindering element and the other is not.

10. The interocular fluid shunting assembly of claim 8, wherein each of the tubes drains interocular fluid to a different location.

11. The interocular fluid shunting assembly of claim 10 wherein the two tubes provide for subconjunctival and suprachoroidal flow.

12. The interocular fluid shunting assembly of claim 11, providing for ab externo suprachoriodal outflow with one tube end accessing the aqueous and other tube end inserted to suprachoroidal space ab externo.
